(19) United States
(12) Patent Application Publication
(10) Pub. No.: US 2026/0000685 A1
(43) Pub. Date: Jan. 1, 2026

(54) PHARMACEUTICAL COMPOSITION FOR PREVENTING OR TREATING DEGENERATIVE BRAIN DISEASES, COMPRISING CANNABINOIDS AS ACTIVE INGREDIENT, AND USES THEREOF

(71) Applicants: NEOCANNBIO CO., LTD., Seoul (KR); KOREA INSTITUTE OF SCIENCE AND TECHNOLOGY, Seoul (KR)

(72) Inventors: Jeong Kook KIM, Seoul (KR); Jung Ho SONG, Seoul (KR); Jungyeob HAM, Gangneung-si (KR); Young-Joo KIM, Gangneung-si (KR); Jin-Chul KIM, Gangneung-si (KR); Taejung KIM, Gangneung-si (KR); Juyong KIM, Gangneung-si (KR)

(73) Assignees: NEOCANNBIO CO., LTD., Seoul (KR); KOREA INSTITUTE OF SCIENCE AND TECHNOLOGY, Seoul (KR)

(21) Appl. No.: 19/111,274

(22) PCT Filed: Sep. 13, 2023
(86) PCT No.: PCT/KR2023/013745

§ 371 Date: Mar. 12, 2025

(30) Foreign Application Priority Data
Sep. 14, 2022 (KR) 10-2022-0115643

(51) Int. Cl. A61K 31/00; A61P 25/28

(52) U.S. Cl. CPC A61K 31/658; A61P 25/28

(57) ABSTRACT

The present disclosure relates to a pharmaceutical composition including cannabinoids as active ingredients, for preventing or treating degenerative brain diseases, and use thereof. The present disclosure has confirmed that when a composition including cannabinoids is used for treatment, a remarkably excellent Alzheimer's treatment or prevention efficacy is exhibited, and thus, the treatment effect on degenerative brain diseases such as Alzheimer's may be enhanced by using the composition.

### TECHNICAL FIELD

The present disclosure relates to a pharmaceutical composition including cannabinoids as active ingredients, for preventing or treating degenerative brain diseases, and use thereof.

### BACKGROUND ART

Cannabis (Cannabis sativa L.) is an annual plant of the genus Cannabis in the family Cannabaceae, which has been widely cultivated in tropical and temperate regions, centered around Central Asia, for over 12,000 years, including wild hemp, and Cannabis chemovars and their variants, including variants var. indica and var. kafiristanica, which contain various kinds of cannabinoid compounds known as medicinal and pharmaceutical ingredients, and Cannabis sativa subspecies sativa, Cannabis sativa subspecies indica, Cannabis sativa subspecies ruderalis, and genetically crossbred, self-bred, or hybrid plants thereof.

In traditional Chinese and Korean medical texts, hulled Cannabis seed, called majain or hwamain, has been used for constipation, diabetes, pain disorders, irregular menstruation, skin diseases, and dysentery, and Cannabis weed which is Cannabis leaf (mayeop) has been used as an anthelmintic, hair protector, asthma, analgesic, anesthetic, diuretic, etc. Additionally, Cannabis root has been used to treat difficult labor and relieve blood stasis, Cannabis skin has been used to treat bruises and open wounds, Cannabis flower has been used to treat paralysis and itching, etc., and Cannabis stem fiber has been used to treat difficult labor, constipation, gout, insanity, insomnia, etc., and there are records of using each part of Cannabis according to the corresponding symptom.

Cannabis includes about 400 compounds, most of which are cannabinoids, terpenes, and phenolic compounds, and of these, there are about 90 cannabinoids, which are medicinally and pharmaceutically important natural ingredients, many of which are ingredients found only in Cannabis. Among the cannabinoids of Cannabis, a psychoactive component is Δ9-tetrahydrocannabinol (Δ9-THC), and cannabidiol (CBD) is a non-psychotropic component known to exhibit physiologically active effects through various receptors in the human body, including adrenergic receptors and cannabinoid receptors.

Meanwhile, as the social environment changes rapidly and diversely, modern people encounter more and more stimulation than in the past, and rapid brain activity is required to accommodate this. Long-term learning or memory activities of adolescents, especially test takers, may cause fatigue in the brain as well as the mind and body, and may also affect the healthy development of the brain. After middle age, symptoms such as memory loss or forgetfulness may appear due to a decline in the function of the central nervous system, and in the elderly, cognitive and memory abilities may decline due to degenerative brain diseases such as Alzheimer's or dementia, and in severe cases, social life itself may become impossible, and considering the impact on family and those around them, enormous social costs may be required.

Most of the drugs currently on the market or in development are drugs that improve the quality of life of Alzheimer's patients by improving their symptoms, including the acetylcholinesterase inhibitors tacrine, donepezil, and rivastigmine, and galantamine, memantine, which has an NMDA-glutamate receptor inhibitory function, etc. However, most of these treatments only provide temporary improvement in clinical symptoms in the early stages of the disease and also cause side effects, making treatment difficult.

Under these circumstances, the present disclosure was completed by confirming a significantly excellent efficacy of a composition including cannabinoids in improving degenerative brain diseases.

### DISCLOSURE OF INVENTION

### Technical Problem

One aspect provides a pharmaceutical composition for preventing or treating a degenerative brain disease, including a cannabinoid as an active ingredient.

### Solution to Problem

One aspect provides a pharmaceutical composition for preventing or treating a degenerative brain disease, including a cannabinoid as an active ingredient.

As used herein, the term “cannabinoid” generally refers to a family of natural products including a 1,1′-di-menthyl-pyrange ring, variously derivatized aromatic rings, and variously unsaturated cyclohexyl rings and their immediate chemical precursors, compounds predominantly found in Cannabis sativa.

The cannabinoid may be acquired from a plant of a genus Cannabis, and specifically, the plant of the genus Cannabis may include Cannabis sp, such as Cannabis chemovars, Cannabis sativa, Cannabis indica, Cannabis ruderalis, etc., and wild seeds, variants, mutations, hybrids, and plants including cannabinoids. Additionally, the Cannabis plant may be a living plant or a dried plant. Additionally, the Cannabis plant may be a leaf, a flower bud, a fruit, a hair, a calyx, a stem, or any part that may contain cannabinoids. Additionally, the Cannabis plant is a monoecious plant, and the cannabinoid content may differ depending on the male and female plants. Plants in the Cannabis genus may be male, female, or a mixture of both.

The cannabinoid may include, for example, one or more of cannabidiol (CBD), cannabidiolic acid (CBDA), cannabinol (CBN), tetrahydrocannabinol (THC), tetrahydrocannabinolic acid (THCA), cannabigerol (CBG), cannabigerolic acid (CBGA), cannabichromene (CBC), cannabichromenic (CBCA), cannabicyclol (CBL), cannabivarin (CBV), cannabidivarin (CBDV), cannabidivarinic (CBDVA), cannabichromevarin (CBCV), cannabigerovarin (CBGV), cannabigerol monomethyl ether (cannabigerol monomethyl ether, CBGM) and cannabielsoin (CBE), cannabicitran (CBT).

Specifically, the cannabinoid may include one or more selected from the group consisting of cannabidiolic acid (CBDA), cannabidiol (CBD), tetrahydrocannabinolic acid (THCA), tetrahydrocannabinol (THC), and cannabinol (CBN), and more specifically, may include one or more selected from the group consisting of CBDA and THCA.

As used herein, the term “Cannabidiol (CBD)” may be represented by the IUPAC name of 2-[(1R,6R)-6-Isopropenyl-3-methylcyclohex-2-en-1-yl]-5-pentylbenzene-1,3-diol and may be represented by the following Formula 1. Additionally, the cannabidiol may include one or more selected from the group consisting of a derivative thereof, a stereoisomer thereof, a pharmaceutically acceptable salt thereof, a hydrate thereof, and a solvate thereof.

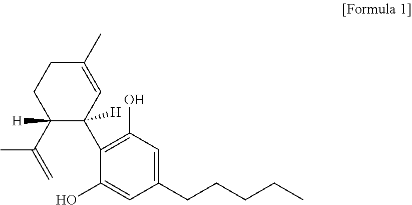 [SMILES: C=C(C)[C@@H]1CCC(C)=C[C@H]1c1c(O)cc(CCCCC)cc1O]

As used herein, the term “Cannabidiolic acid (CBDA)” may be represented by the IUPAC name of (1′R,2′R)-2,6-Dihydroxy-5-methyl-4-pentyl-2′-(prop-1-en-2-yl)-1,2,3,4-tetrahydro[1,1′-biphenyl]-3-carboxylic acid, and may be expressed by the following Formula 2. Additionally, the cannabidiolic acid may include one or more selected from the group consisting of a derivative thereof, a stereoisomer thereof, a pharmaceutically acceptable salt thereof, a hydrate thereof, and a solvate thereof.

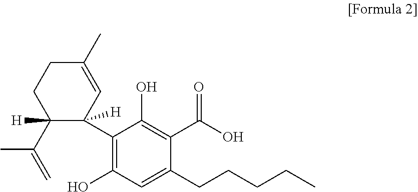 [SMILES: C=C(C)[C@@H]1CCC(C)=C[C@H]1c1c(O)cc(CCCCC)c(C(=O)O)c1O]

As used herein, the term “Tetrahydrocannabinol (THC)” may refer to Δ⁹-tetrahydrocannabinol (Δ⁹-THC) or Δ⁸-tetrahydrocannabinol (Δ⁸-THC). The Δ⁹-tetrahydrocannabinol may be represented by the IUPAC name of (6aR, 10aR)-delta-9-Tetrahydrocannabinol and may be expressed by the following Formula 3. The Δ⁸ tetrahydrocannabinol may be represented by the IUPAC name of 6,6,9-trimethyl-3-pentyl-6a,7,10,10a-tetrahydrobenzo[c]chromen-1-ol and may be expressed by the following Formula 4. The tetrahydrocannabinol may include one or more of Δ⁹-tetrahydrocannabinol and Δ⁸-tetrahydrocannabinol, and specifically may include Δ⁹-tetrahydrocannabinol. Additionally, the tetrahydrocannabinol may include one or more selected from the group consisting of a derivative thereof, a stereoisomer thereof, a pharmaceutically acceptable salt thereof, a hydrate thereof, and a solvate thereof.

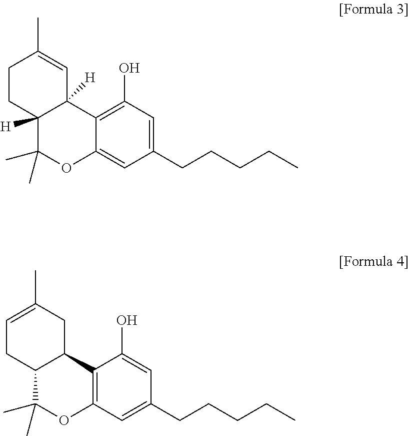

As used herein, the term “Tetrahydrocannabinolic acid (THCA)” may refer to Δ⁹-Tetrahydrocannabinolic acid (Δ⁹-THCA) or Δ⁸-Tetrahydrocannabinolic acid (Δ⁸-THCA). The Δ⁹-tetrahydrocannabinolic acid may be represented by the IUPAC name of (6aR, 10aR)-1-Hydroxy-6,6,9-trimethyl-3-pentyl-6a,7,8,10a-tetrahydro-6H-benzo[c]chromene-2-carboxylic acid and may be expressed by the following Formula 5. The Δ⁸-tetrahydrocannabinolic acid may be represented by the IUPAC name of 6,6,9-trimethyl-3-pentyl-6a,7,10,10a-tetrahydrobenzo[c]chromen-1-ol and may be expressed by the following Formula 6. The tetrahydrocannabinolic acid may include one or more one of Δ⁹-tetrahydrocannabinolic acid and Δ⁸-tetrahydrocannabinolic acid, and specifically may include Δ⁹-tetrahydrocannabinolic acid. Additionally, the tetrahydrocannabinolic acid may include one or more selected from the group consisting of a derivative thereof, a stereoisomer thereof, a pharmaceutically acceptable salt thereof, a hydrate thereof, and a solvate thereof.

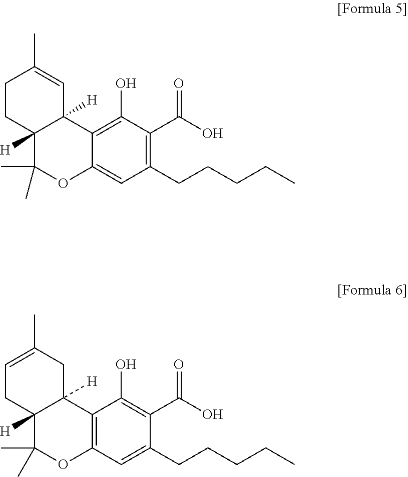

As used herein, the term “Cannabinol (CBN)” may be represented by the IUPAC name of 6,6,9-Trimethyl-3-pentyl-benzo[c]chromen-1-ol and may be expressed by the following Formula 7. Additionally, the cannabinol may include one or more selected from the group consisting of a derivative thereof, a stereoisomer thereof, a pharmaceutically acceptable salt thereof, a hydrate thereof, and a solvate thereof.

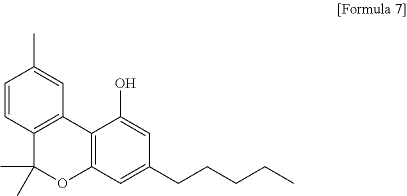 [SMILES: CCCCCc1cc(O)c2c(c1)OC(C)(C)c1ccc(C)cc1-2]

As used herein, the term “pharmaceutically acceptable salt” refers to a form of a salt that may be used pharmaceutically among salts, which are substances in which cations and anions are bonded by electrostatic attraction, and may typically be a metal salt, a salt with an organic base, a salt with an inorganic acid, a salt with an organic acid, a salt with a basic or acidic amino acid, etc. For example, the metal salts may be alkali metal salts (sodium salt, potassium salt, etc.), alkaline earth metal salts (calcium salt, magnesium salt, barium salt, etc.), aluminum salts, etc.; the salts with organic bases may be salts with triethylamine, pyridine, picoline, 2,6-lutidine, ethanolamine, diethanolamine, triethanolamine, cyclohexylamine, dicyclohexylamine, N,N-dibenzylethylenediamine, etc.; the salts with inorganic acids may be salts with hydrochloric acid, hydrobromic acid, nitric acid, sulfuric acid, phosphoric acid, etc.; the salts with organic acids may be salts with formic acid, acetic acid, trifluoroacetic acid, phthalic acid, fumaric acid, oxalic acid, tartaric acid, maleic acid, citric acid, succinic acid, methanesulfonic acid, benzenesulfonic acid, p-toluenesulfonic acid, etc., salts with basic amino acids may be salts with arginine, lysine, ornithine, etc.; salts with acidic amino acids may be salts with aspartic acid, glutamic acid, etc. Particularly preferable salts are, when the compound has an acidic functional group therein, inorganic salts such as alkali metal salts (for example, sodium salts, potassium salts, etc.), alkaline earth metal salts (for example, calcium salts, magnesium salts, barium salts, etc.), etc., and organic salts such as ammonium salts; and, when the compound has a basic functional group therein, salts with inorganic acids such as hydrochloric acid, hydrobromic acid, nitric acid, sulfuric acid, phosphoric acid, etc., and salts with organic acids such as acetic acid, phthalic acid, fumaric acid, oxalic acid, tartaric acid, maleic acid, citric acid, succinic acid, methanesulfonic acid, p-toluenesulfonic acid, etc.

The cannabinoids may include cannabidiolic acid (CBDA) and tetrahydrocannabinolic acid (THCA), and the pharmaceutical composition for preventing or treating degenerative brain diseases may include CBDA and THCA. Additionally, the composition may include CBDA and THCA at a certain concentration ratio.

The concentration ratio of CBDA and THCA (CBDA:THCA) included in the composition may be from 10:0 (CBDA alone) to 7:3, and specifically, may be included at a concentration ratio, 10:0 to 7:3, 10:0 to 7.5:2.5, 10:0 to 8:2, 10:0 to 8.5:1.5, 10:0 to 9:1, 10:0 to 9.5:0.5, 9.5:0.5 to 7:3, 9.5:0.5 to 7.5:2.5, 9.5:0.5 to 8:2, 9.5:0.5 to 8.5:1.5, 9.5:0.5 to 9:1, 9:1 to 7:3, 9:1 to 7.5:2.5, 9:1 to 8:2, 9:1 to 8.5:1.5, 8.5:1.5 to 7:3, 8.5:1.5 to 7.5:2.5, 8.5:1.5 to 8:2, 8:2 to 7:3 or 8:2 to 7.5:2.5.

Furthermore, the concentration ratio of THCA and CBDA (THCA:CBDA) included in the composition may be from 10:0 (THCA alone) to 7:3, and specifically, may be included at a concentration ratio, 10:0 to 7:3, 10:0 to 7.5:2.5, 10:0 to 8:2, 10:0 to 8.5:1.5, 10:0 to 9:1, 10:0 to 9.5:0.5, 9.5:0.5 to 7:3, 9.5:0.5 to 7.5:2.5, 9.5:0.5 to 8:2, 9.5:0.5 to 8.5:1.5, 9.5:0.5 to 9:1, 9:1 to 7:3, 9:1 to 7.5:2.5, 9:1 to 8:2, 9:1 to 8.5:1.5, 8.5:1.5 to 7:3, 8.5:1.5 to 7.5:2.5, 8.5:1.5 to 8:2, 8:2 to 7:3 or 8:2 to 7.5:2.5.

As used herein, the term “degenerative brain disease” refers to a disease occurring in the brain among degenerative diseases that occur with age, and is a concept that includes all diseases that may exhibit symptoms such as decline or decrease in cognitive ability or memory ability.

The degenerative brain disease may be a degenerative brain disease mediated by hyperphosphorylation of tau protein; and/or overexpression, aggregation or deposition, etc., of amyloid beta.

The degenerative brain disease may include one or more selected from the group consisting of dementia with Lewy Bodies (DLB), multi-infarct dementia (MID), frontotemporal lobar degeneration (FTLD), pick's disease, corticobasal degeneration (CBD), progressive supranuclear palsy (PSP), Parkinson's disease, Alzheimer's disease, Huntington's disease, amnesia and memory impairment, and may be specifically Alzheimer's disease or dementia, and more specifically Alzheimer's disease.

As used herein, the term “Alzheimer's disease” is a degenerative neurological disease in which abnormal proteins (amyloid beta protein, tau protein) accumulate in the brain, causing brain neuronal cells to gradually die. Alzheimer's disease is the most common cause of dementia, and it is known that approximately 50-60% of all dementia patients show symptoms of dementia caused by Alzheimer's disease. One of the pathological hallmarks of Alzheimer's disease is an accumulation of senile plaques on the outside of neuronal cells, the causative agent of which is amyloid beta (β-amyloid, Aβ). Amyloid beta (Aβ) is a protein fragment cut from amyloid precursor protein (APP), and if a large amount of amyloid beta is produced due to abnormal metabolism of amyloid precursor protein, it may cause brain cell toxicity and lead to Alzheimer's disease.

In the composition including the CBDA and THCA, the CBDA and THCA may be co-administered, and specifically, the CBDA and THCA may be administered simultaneously in one formulation, or the CBDA and THCA may be administered simultaneously or sequentially in separate formulations.

As used herein, the term “co-administration” may be achieved by administering individual ingredients of a treatment regimen simultaneously, sequentially, or individually. Co-administration is a method of obtaining a combination treatment effect by administering two or more drugs simultaneously or sequentially, or alternately at regular or indefinite intervals, etc., concomitant therapy is not limited thereto, but may be defined as a method in which the efficacy measured through the level of response, the rate of response, the period until disease progression, or the survival period is therapeutically superior to the efficacy that may be obtained by administering one or the remaining ingredients of the concomitant therapy at a typical dose and may provide a synergistic effect.

As used herein, the term “treatment” refers to any action that improves or beneficially changes the symptoms of degenerative brain disease by administering the composition of the present disclosure.

As used herein, the term “prevention” refers to any action by which the possibility of developing degenerative brain disease or a disease is inhibited or delayed by administration of the composition of the present disclosure.

The composition may exhibit one or more characteristics selected from the following: (a) inhibition of activation or phosphorylation of tau protein; (b) inhibition of expression, aggregation, or deposition of amyloid beta (Aβ); (c) inhibition of intracellular Ca²⁺ dyshomeostasis or maintenance of Ca²⁺ homeostasis; (d) inhibition of nerve or neuronal cell damage; (e) enhancement/induction of the expression level or activity of brain-derived neurotrophic factor (BDNF); (f) enhancement/induction of activation or phosphorylation of CAMP response element-binding protein (CREB); and (g) enhancement/induction of activation or phosphorylation of tyrosine receptor kinase B (TrkB).

According to an embodiment, it was confirmed that the expression of amyloid beta (Aβ) and phosphorylated tau protein (p-Tau) was inhibited, the concentration of abnormal calcium ions was reduced to normal, and the expression levels of BDNF, p-CREB and p-TrKB were increased by treatment of a composition including the cannabinoid in neuronal cells damaged by Aβ1-42 or in hippocampal tissue of an Alzheimer's animal model treated with Aβ1-42, thereby recovering to a level similar to that of undamaged control group neuronal cells or hippocampal tissue of an animal.

In addition, according to an embodiment, when a composition including the cannabinoid was treated in an Alzheimer's animal model treated with Aβ1-42, it was confirmed that the results of cognitive and memory ability evaluations in the Morris water maze test, novel object recognition test, and object location test were restored to a level similar to that of the control group animal.

Therefore, based on the results, it may be seen that a composition including cannabinoids, specifically a composition including CBDA and/or THCA, may exhibit excellent effects in the treatment or prevention of degenerative brain diseases including Alzheimer's disease and dementia.

The pharmaceutical composition may include a pharmaceutically acceptable carrier. The “pharmaceutically acceptable carrier” may refer to a carrier or diluent that does not stimulate an organism and does not inhibit the biological activity and properties of a compound being injected. Here, “pharmaceutically accepted” means that it does not inhibit the activity of the active ingredient and does not have any toxicity beyond what the subject of application (prescription) may adapt to. Any type of carrier that may be used in the pharmaceutical composition may be used as long as the carrier is a pharmaceutically accepted carrier commonly used in the art. Non-limiting examples of the carrier may be lactose, dextrose, maltodextrin, sucrose, sorbitol, mannitol, xylitol, erythritol, maltitol, glycerol, ethanol, starch, acacia gum, alginate, gelatin, calcium phosphate, calcium silicate, cellulose, methyl cellulose, microcrystalline cellulose, polyvinyl pyrrolidone, water, saline, sterile water, Ringer's solution, buffered saline, albumin injection solution, methylhydroxybenzoate, propylhydroxybenzoate, talc, magnesium stearate, or mineral oil, etc. They may be used alone or in combination of two or more. The pharmaceutical composition may be prepared as an oral formulation or a parenteral formulation depending on an administration route by a typical method known in the art, including a pharmaceutically accepted carrier in addition to the active ingredient. The pharmaceutical composition may be formulated and used in the form of an oral formulation such as a pill, granule, tablet, capsule, suspension, emulsion, syrup, aerosol, etc., topical preparation, suppository, or sterile injectable solution, each according to typical methods.

The pharmaceutical composition may be formulated and used in the form of an oral formulation such as a pill, granule, tablet, capsule, suspension, emulsion, syrup, aerosol, etc., topical preparation, suppository, or sterile injectable solution, each according to typical methods. When formulating the pharmaceutical composition, the pharmaceutical composition may be prepared by adding diluents or excipients such as commonly used filler, bulking agent, binder, humectant, disintegrant, or surfactant, etc.

When the pharmaceutical composition is prepared as an oral formulation, the pharmaceutical composition may be prepared in the form of a powder, granule, tablet, pill, dragee, capsule, liquid, gel, syrup, suspension, wafer, etc., along with a suitable carrier, according to methods known in the art. Examples of pharmaceutically accepted suitable carriers may be sugars such as lactose, glucose, sucrose, dextrose, sorbitol, mannitol, and xylitol, etc., starches such as corn starch, potato starch, and wheat starch, etc., celluloses such as cellulose, methylcellulose, ethylcellulose, sodium carboxymethylcellulose, and hydroxypropylmethylcellulose, etc., polyvinyl pyrrolidone, water, methylhydroxybenzoate, propylhydroxybenzoate, magnesium stearate, mineral oil, malt, gelatin, talc, polyols, and vegetable oils. When formulating, the formulation may include diluents and/or excipients such as a filler, bulking agent, binder, humectant, disintegrant, and surfactant, etc. as needed.

When the pharmaceutical composition is prepared as a parenteral formulation, the pharmaceutical composition may be formulated into the form of an injection, a transdermal administration agent, a nasal inhalant, and suppository along with a suitable carrier according to methods known in the art. When formulated as an injectable formulation, suitable carriers may include sterile water, ethanol, polyols such as glycerol or propylene glycol, or mixtures thereof, desirably Ringer's solution, phosphate buffered saline (PBS) containing triethanolamine, sterile water for injection, and isotonic solutions such as 5% dextrose, etc. may be used. When formulated as a transdermal agent, the transdermal agent may be formulated in the form of an ointment, cream, lotion, gel, topical solution, paste, liniment, and aerosol, etc. For a nasal inhalant, the nasal inhalant may be formulated in the form of an aerosol spray using a suitable propellant such as dichlorofluoromethane, trichlorofluoromethane, dichlorotetrafluoroethane, carbon dioxide, etc., when formulated as a suppository, the base used may be witepsol, tween 61, polyethylene glycol, cacao butter, laurin butter, polyoxyethylene sorbitan fatty acid esters, polyoxyethylene stearate, sorbitan fatty acid esters, etc.

The pharmaceutical composition may be administered in a pharmaceutically effective amount, wherein the term “pharmaceutically effective amount” means an amount sufficient to treat or prevent a disease at a reasonable benefit/risk ratio applicable to medical treatment or prevention, and the effective dosage level may be determined depending on the severity of the disease, the activity of the drug, the patient's age, weight, health, sex, the patient's sensitivity to the drug, the time of administration, the route of administration and the excretion rate of the composition of the present disclosure used, the treatment period, the drug used in combination with or concurrently with the composition of the present disclosure used, and other factors well known in the medical field. The pharmaceutical composition may be administered alone or in combination with an ingredient known to exhibit a therapeutic effect on a known degenerative brain disease. Taking all of the factors into account, it is important to administer the minimum amount that may achieve the maximum effect without side effects.

The dosage of the pharmaceutical composition may be determined by a person skilled in the art considering the purpose of use, the level of addiction of the disease, the patient's age, weight, gender, pre-existing condition, or the type of substance used as an active ingredient, etc. For example, the pharmaceutical composition of the present disclosure may be administered at a dose of from about 0.1 ng to about 1,000 mg/kg per adult, desirably from 1 ng to about 100 mg/kg, and the frequency of administration of the compositions of the present disclosure is not specifically limited, but may be administered once daily or administered multiple times in divided doses. The dosage or frequency of administration does not limit the scope of the present disclosure in any way.

Another aspect provides a method of treating or preventing a degenerative brain disease, including administering to a subject a cannabinoid or a pharmaceutical composition for preventing or treating the degenerative brain disease. The same aspects as described above apply equally to the method.

As used herein, the term “subject” may include, without limitation, mammals, birds, reptiles, farmed fish, etc., including mice, livestock, humans, etc., that develop or are at risk of developing degenerative brain disease, and the subject may exclude humans.

The pharmaceutical composition may be administered in single or multiple doses in a pharmaceutically effective amount. At this time, the composition may be formulated and administered in the form of a liquid, powder, aerosol, injection, infusion (Ringel), capsule, pill, tablet, suppository, or patch. The administration route of the pharmaceutical composition for preventing or treating degenerative brain disease may be by any common route as long as the pharmaceutical composition may reach the target tissue.

The pharmaceutical composition may be administered by routes such as, but is not particularly limited to, intraperitoneal administration, intravenous administration, intramuscular administration, subcutaneous administration, intradermal administration, transdermal patch administration, oral administration, intranasal administration, intrapulmonary administration, or rectal administration, etc., depending on the intended purpose. However, when administered by oral administration, the pharmaceutical composition may be administered in an unformulated form, and since the active ingredient of the pharmaceutical composition may be denatured or decomposed by gastric acid, the oral composition may be administered orally in a form that coats the active agent or is formulated to protect the active agent from decomposition in the stomach, or in the form of an oral patch. Additionally, the composition may be administered by any device that may transport the active substance to target cells.

Another aspect provides a use of cannabinoids or compositions including them as active ingredients for the prevention or treatment of degenerative brain diseases. The same aspects as described above apply equally to the method.

### Advantageous Effects of Invention

The present disclosure has confirmed that when a composition including cannabinoids is treated, a remarkably excellent Alzheimer's treatment or prevention efficacy is exhibited, and thus, the treatment effect of degenerative brain diseases such as Alzheimer's, etc., may be enhanced by using the composition.

### BRIEF DESCRIPTION OF DRAWINGS

FIG. 1 is a diagram illustrating the survival rates of primary neuronal cells according to treatment with different concentrations of cannabinoids.

FIG. 2 is a diagram illustrating inhibitory efficacies of primary neuronal cell apoptosis according to treatment with different concentrations of cannabinoids.

FIG. 3 is a diagram illustrating an inhibitory efficacies of primary neuronal cell apoptosis according to treatment with the same concentrations of cannabinoids.

FIG. 4 is a diagram illustrating the inhibitory efficacies of primary neuronal cell apoptosis according to combination ratios of CBDA and THCA.

FIG. 5 is a diagram illustrating the level of APP/AB protein expression in primary neuronal cells according to treatment with cannabinoids (CBDA, THCA, THC, and CBN).

FIG. 6 is a diagram illustrating the expression levels of Amyloid Precursor Protein (APP), polymeric Aβ, and oligomeric Aβ in primary neuronal cells according to treatment with cannabinoids (CBDA and THCA).

FIG. 7 is a diagram illustrating the expression levels of Tau/p-Tau protein and phosphorylation levels in primary neuronal cells according to treatment with cannabinoids (CBDA, THCA, THC, and CBN).

FIG. 8 is a diagram illustrating the results of microscopic observation of Ca²⁺ levels in primary neuronal cells according to treatment with cannabinoids (CBDA, CBD, and THCA).

FIG. 9 is a diagram illustrating the Ca²⁺ concentration of primary neuronal cells according to treatment with cannabinoids (CBDA, CBD, and THCA).

FIG. 10 is a diagram illustrating an experimental schedule for producing an Alzheimer's animal model and conducting cognitive behavioral analysis.

FIGS. 11 to 13 are diagrams illustrating the results of a Morris water maze test of animal models treated with cannabinoids (CBDA and THCA).

FIG. 14 is a diagram illustrating the results of a novel object recognition test and an object location test in animal models treated with cannabinoids (CBDA and THCA).

FIG. 15 is a diagram illustrating expression levels of APP/AB protein in the hippocampus of animal models treated with cannabinoids (CBDA and THCA).

FIG. 16 is a diagram illustrating the expression levels and phosphorylation levels of Tau/p-Tau protein in the hippocampus of animal models treated with cannabinoids (CBDA and THCA).

FIG. 17 is a diagram illustrating the expression levels and phosphorylation levels of BDNF, p-CREB, and p-trkb proteins in the hippocampus of animal models treated with cannabinoids (CBDA and THCA).

#p<0.05, ##p<0.005, and ###p<0.001 vs. PBS treated group (control group). * p<0.05, ** p<0.005, and *** p<0.001 vs. Aβ1-42 treated group.

### MODE FOR THE INVENTION

The present disclosure will be explained in more detail in the following embodiments. However, these embodiments are for illustrative purposes only and the scope of the disclosure is not limited to these embodiments.

### EXAMPLE 1: CANNABINOID INFORMATION

The Cannabis-derived compounds used in the following examples were manufactured based on the Seoul Regional Food and Drug Safety Office's Narcotics (Cannabis) Academic Researcher's Permit (No. 1564, No. 1979, and No. 2083), and information on the six selected cannabinoid compounds is disclosed in Table 1 below.

TABLE 1

| | | Concen- | |
|---|---|---|---|
| | | tration | Volume |
| Compound | Sample Name | (mM) | (μL) |
| CBDA (Cannabidiolic acid) | KSAM001 (001) | 10 | 90 |
| CBD (Cannabidiol) | KSAM002 (002) | 10 | 90 |
| THCA (Delta 9- | KSAM003 (003) | 10 | 90 |
| Tetrahydrocannabinolic acid) | | | |
| THC (Delta 9- | KSAM004 (004) | 10 | 90 |
| Tetrahydrocannabinol) | | | |
| CBN (Cannabinol) | KSAM005 (005) | 10 | 90 |
| Synthetic CBN | KSAM006 (006) | 10 | 90 |

### EXAMPLE 2: EVALUATION OF NEUROTOXICITY OF CANNABINOIDS

To evaluate the toxicity of the six compounds acquired in Example 1 above to neuronal cells, the following experiments were conducted.

Specifically, primary cortical neuronal cells were used as neuronal cells, and the primary neuronal cells were acquired by culturing in vitro the cerebral cortex tissue obtained by dissecting a 15-day embryonic ICR mouse. Next, the primary neuronal cells were seeded in 96-wells at 5×10⁵ cells per well and cultured for 6 days. Afterwards, the six compounds of Example 1 above were treated at different concentrations for 24 hours. Afterwards, a MTS solution was added to the medium and incubated for 2 hours. Cytotoxicity (cell viability) was measured using a microplate spectrophotometer (Bio-Tek Power Wave XS, Winooski, VT, USA), and absorbance was measured at 490 nm.

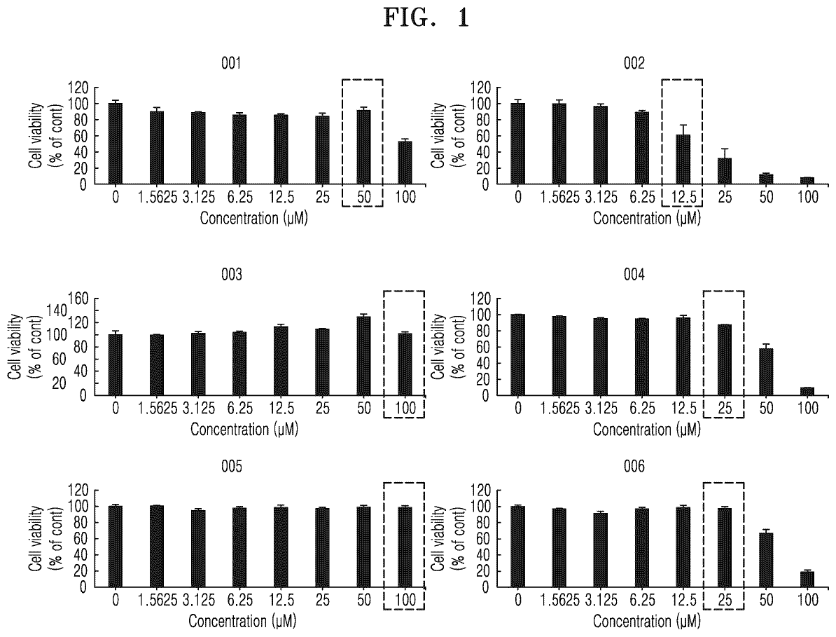

As a result, it was confirmed that each compound exhibited different levels of cytotoxicity, indicating that the properties for neuronal cells differ depending on the type of compound (FIG. 1). Additionally, in the examples below, experiments were conducted with compounds at concentrations that did not exhibit toxicity to neuronal cells.

### EXAMPLE 3: EVALUATION OF THE INHIBITORY EFFICACY OF CANNABINOIDS ON NEURON APOPTOSIS

To evaluate the neuron apoptosis inhibition efficacy of the six compounds acquired in Example 1 above, the following experiments were conducted.

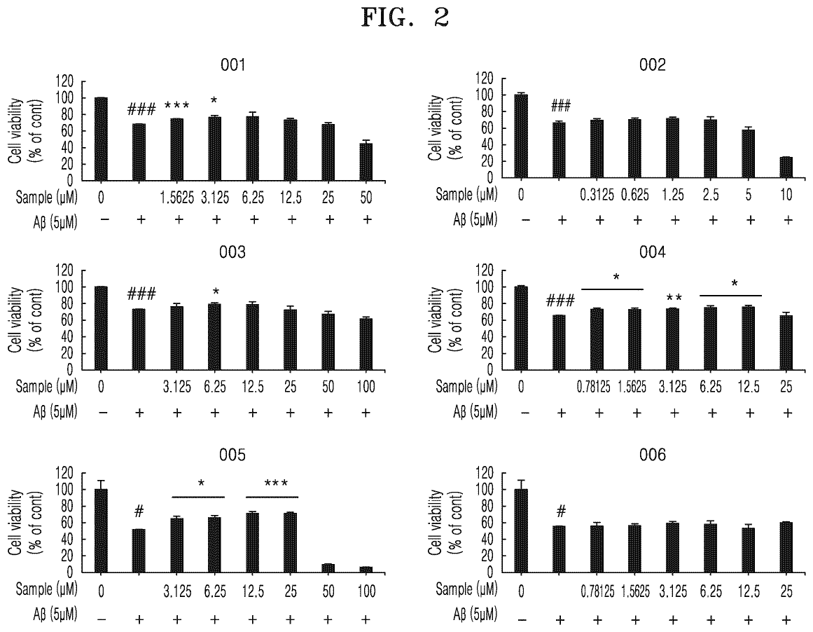

Specifically, primary neuronal cells derived from ICR mice were seeded in 96-wells at 5×10⁵ cells per well and cultured for 6 days. Afterwards, the six compounds of Example 1 above were treated at different concentrations and Aβ1-42 (5 μM) was treated to induce neuron apoptosis, and the cells were treated together for 24 hours. Afterwards, a MTS solution was added to the medium and incubated for 2 hours. Cytotoxicity (cell viability) was measured using a microplate spectrophotometer (Bio-Tek Power Wave XS, Winooski, VT, USA), and absorbance was measured at 490 nm. As a result, it was confirmed that the cell survival rate level significantly increased in neuronal cells treated with CBDA, THCA, THC, or CBN compared to the control group treated with Aβ1-42 alone (FIG. 2). Therefore, it may be seen that among the compounds derived from the Cannabis extract, CBDA, THCA, THC, and CBN have excellent neuroprotective effects.

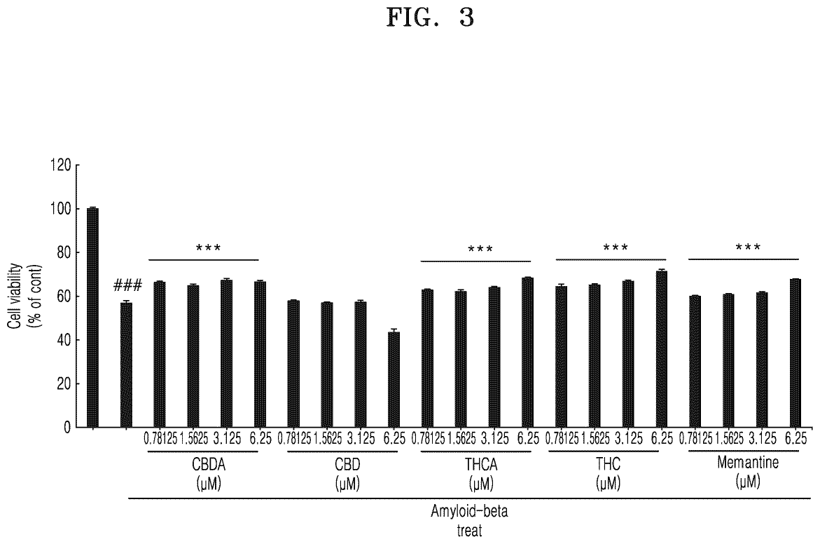

Next, the neuron apoptosis inhibition efficacy for the same concentration of CBDA, CBD, THCA and THC was evaluated using the same method as above. Memantine, known as an Alzheimer's treatment, was used as a positive control group. As a result, it was confirmed that CBDA and THCA had better neuron apoptosis inhibition efficacy (FIG. 3).

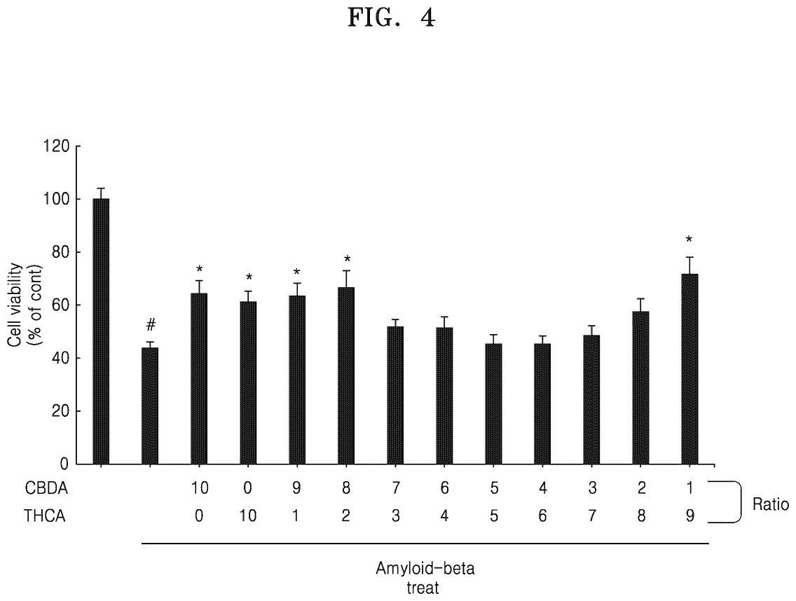

Next, the neuron apoptosis inhibition efficacy according to the combination ratio of CBDA and THCA, which were confirmed to have excellent efficacy above, was evaluated using the same method as above. As a result, it was confirmed that CBDA:THCA exhibited excellent inhibitory efficacy when mixed at a specific ratio, and in particular, it was confirmed that an excellent effect was exhibited when CBDA:THCA was 9:1 to 8:2 (FIG. 4).

### EXAMPLE 4: EVALUATION OF EFFICACY OF CANNABINOIDS IN INHIBITING AMYLOID BETA (Aβ) EXPRESSION LEVELS

To evaluate the inhibitory efficacy of four compounds (CBDA, THCA, THC, and CBN) that exhibited excellent effects in Example 3 above on amyloid beta (Aβ), an Alzheimer's disease pathology marker, the following experiments were conducted.

Specifically, primary neuronal cells derived from ICR mice were cultured for 6 days, and then treated with CBDA, THCA, THC, or CBN compounds while treating with Aβ1-42 (5 μM), and treated together for 24 hours. Next, Western blotting was conducted to confirm the expression levels of APP (Amyloid Precursor Protein), polymeric Aβ, and oligomeric AB in the cells.

To conduct Western blotting, tissues were homogenized in radioimmunoprecipitation assay buffer (Cell Signaling) to acquire tissue lysates. Tissue protein concentrations were determined using the Bio-Rad protein assay (Bio-Rad, Hercules, CA, USA), and Western blot analysis was conducted using 80-120 μg of protein. Next, the Western blotting samples prepared using the proteins were separated using 12% SDS-PAGE and transferred to a polyvinylidene fluoride membrane (PVDF) (Merck Millipore, Burlington, MA, USA; 0.4 μm). The PVDF was blocked with tris-buffered saline including 5% bovine serum albumin and Tween-20, and incubated with primary antibodies overnight at 4° C. After primary antibody incubation, the samples were incubated with HRP-conjugated secondary antibody (Sigma; 1:5000) for 1 hour at room temperature, and immunodetection was conducted using an enhanced chemiluminescence detection kit (GE Healthcare, Chicago, IL, USA).

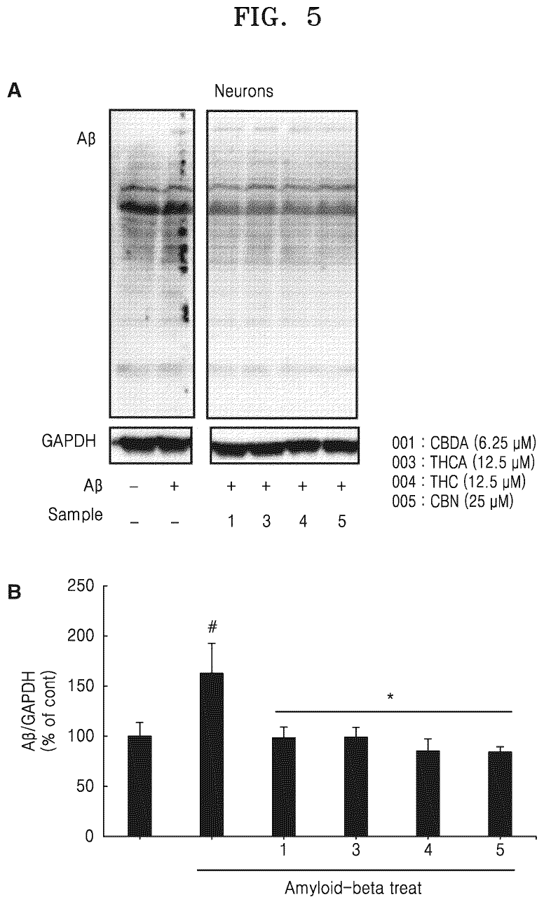

As a result, compared to the control group treated with Aβ1-42 alone, it was confirmed that the expression level of AB was significantly reduced in neuronal cells treated with CBDA, THCA, THC, or CBN (FIG. 5).

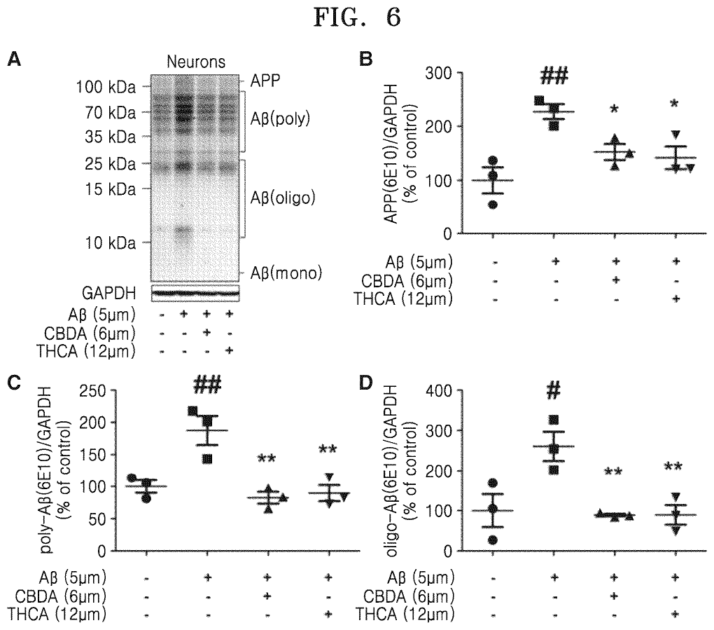

In addition, when the expression levels of APP (Amyloid Precursor Protein), polymeric Aβ, and oligomeric AB were confirmed by treating CBDA (6 μM) or THCA (5 μM) compounds in the same manner as above, it was confirmed that the expression levels of APP, polymeric Aβ, and oligomeric Aβ were significantly reduced in neuronal cells treated with CBDA or THCA (FIG. 6).

Therefore, it may be seen that CBDA, THCA, THC, and CBN may effectively inhibit Aβ, an Alzheimer's pathology marker.

### EXAMPLE 5: EVALUATION OF PHOSPHORYLATED TAU (P-TAU) EXPRESSION LEVEL AND PHOSPHORYLATION INHIBITION EFFICACY OF CANNABINOIDS

To evaluate the efficacy of four compounds (CBDA, THCA, THC, and CBN) that exhibited excellent effects in Example 3 above on the expression and phosphorylation inhibition of phosphorylated tau (p-Tau), an Alzheimer's disease pathology marker, the following experiments were conducted.

Specifically, primary neuronal cells derived from ICR mice were cultured for 6 days, and then treated with CBDA, THCA, THC, or CBN compounds while treating with Aβ1-42 (5 μM), and treated together for 24 hours. Next, to confirm the expression levels of Tau and phosphorylated Tau (p-Tau) in the cells, Western blotting was conducted using the method disclosed in Example 4 above.

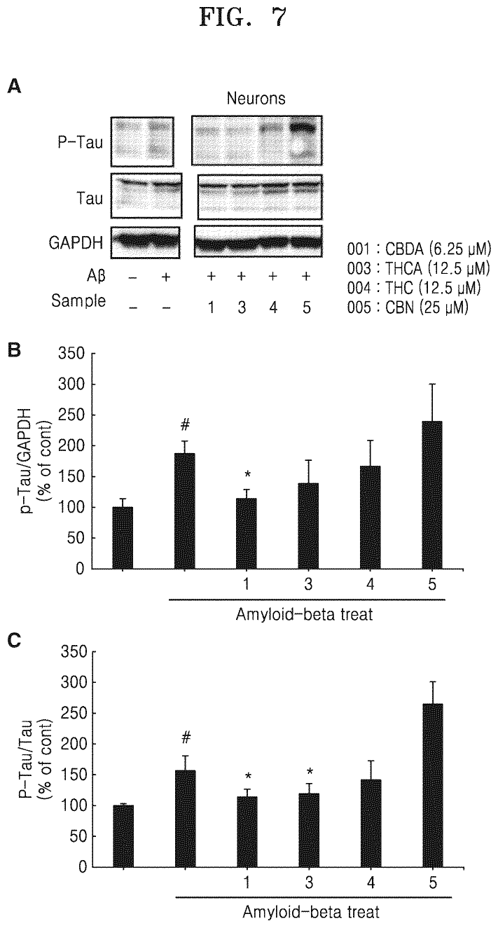

As a result, compared to the control group treated with Aβ1-42 alone, it was confirmed that the expression level and phosphorylation level of p-Tau were significantly reduced in the neuronal cells treated with CBDA, THCA, or THC, but not in the cells treated with CBN (FIG. 7).

Therefore, it may be seen that CBDA, THCA and THC may effectively inhibit p-Tau, an Alzheimer's pathology marker.

### EXAMPLE 6: EVALUATION OF EFFICACY OF CANNABINOIDS IN REGULATING CALCIUM CONCENTRATION IN NEURONAL CELLS

To evaluate the efficacy of the three compounds (CBDA, THCA, and THC) that exhibited excellent effects in Example 5 above in regulating intracellular calcium concentration in neuronal cells, the following experiments were conducted.

Specifically, primary cortical neuron cells were seeded in 96-wells at 5×10⁵ cells per well and cultured for 6 days. Then, the three compounds CBDA (6.25 μM), THCA (12.5 μM), and THC (12.5 μM) that exhibited excellent efficacy in Example 5 above were treated, and Aβ1-42 (5 μM) was treated together for 24 hours. Next, to measure the concentration of calcium ions in neuronal cells by detecting Ca²⁺ using fluorescence imaging, the cells were washed with PBS and treated with 10 μM Ca²⁺ indicator fluo-4 AM (Thermo) for 24 hours. The stained sections were washed with PBS, covered with Prolong Gold Antifide Reagent (Invitrogen, Carlsbad, CA, USA) including DAPI nuclear stain, and observed using a microscope (Carl Zeiss, Oberkochen, Germany). Fluo-4 AM fluorescence signal intensity was measured using the Image J analysis program (National Institute of Health, Bethesda, MA, USA).

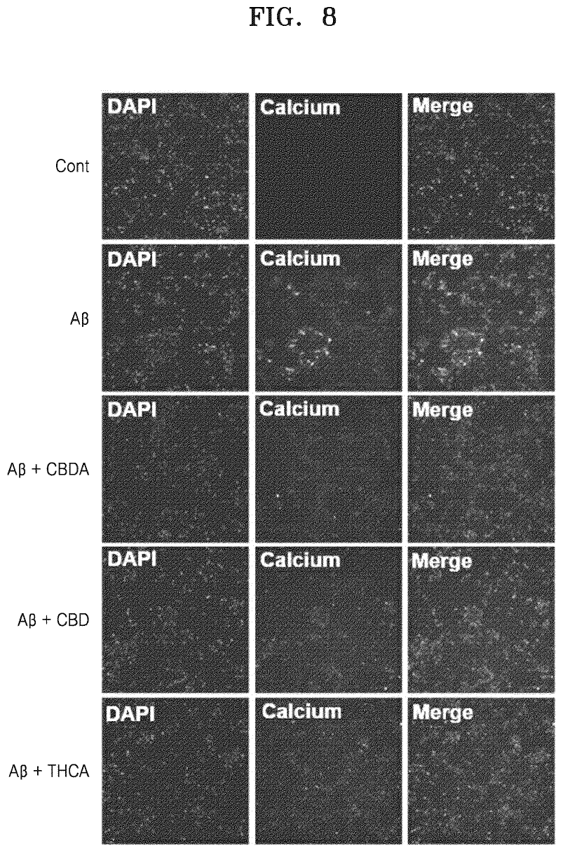

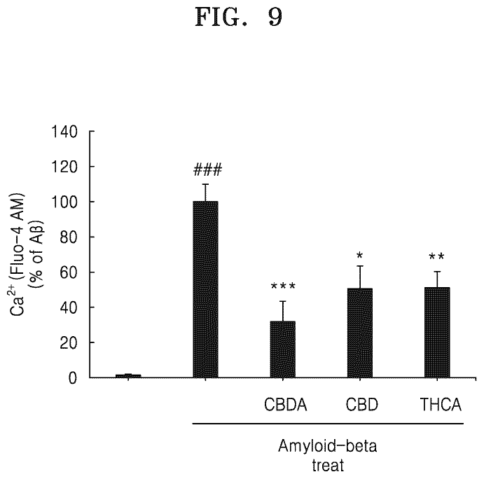

As a result, compared to the control group treated with Aβ1-42 alone, it was confirmed that the intracellular calcium ion concentration was significantly reduced in neuronal cells treated with CBDA, THCA, or THC (FIG. 8 and FIG. 9). Therefore, it may be seen that CBDA, THCA and THC may inhibit calcium homeostasis abnormalities by effectively regulating the intracellular concentration of calcium ions in neuronal cells.

### EXAMPLE 7: EVALUATION OF COGNITIVE-BEHAVIORAL INDICATORS FOLLOWING CANNABINOID TREATMENT

To evaluate the efficacy of Cannabis-derived compounds on the treatment of Alzheimer's disease and on indicators of cognitive and memory function, the following experiments were conducted.

### 7-1: Animal Model Creation and Experimental Planning

Female ICR mice (8 weeks old) used in the experiment were purchased from Coretech (Pyeongtaek, Korea). Mice were housed in the animal care facility of the Korea Institute of Science and Technology (KIST) (temperature 22±2° C., humidity 40-60%, 12-hour light/dark cycle). Mice were housed in an environment with free access to food and water. All animal experiments were conducted in accordance with the protocol approval of the Animal Ethics Committee of our institution.

Next, to create an animal model, mice were randomly selected and divided into four groups as follows: PBS treated group (PBS+PBS), Aβ1-42 treated group (Aβ1-42 +PBS) (Alzheimer's animal model), CBDA treated group (Δβ1-42+CBDA (6.25 UM, 3 μL/mouse)), and THCA treated group (Δβ1-42+THCA (12.5 μM, 3 μL/mouse)).

After anesthetizing the isolated mice by intraperitoneal (i.p.) injection of avertin (250 mg/kg), the mice were fixed in a stereotaxic apparatus, and CBDA, THCA, Aβ1-42, or PBS (3 μl/15 min/mouse) were gradually delivered to the hippocampus using a Hamilton syringe (coordinates of bregma: mediolateral (ML)=1.30 mm, anteroposterior (AP)=−2.00 mm, dorsoventral=−2.20 mm).

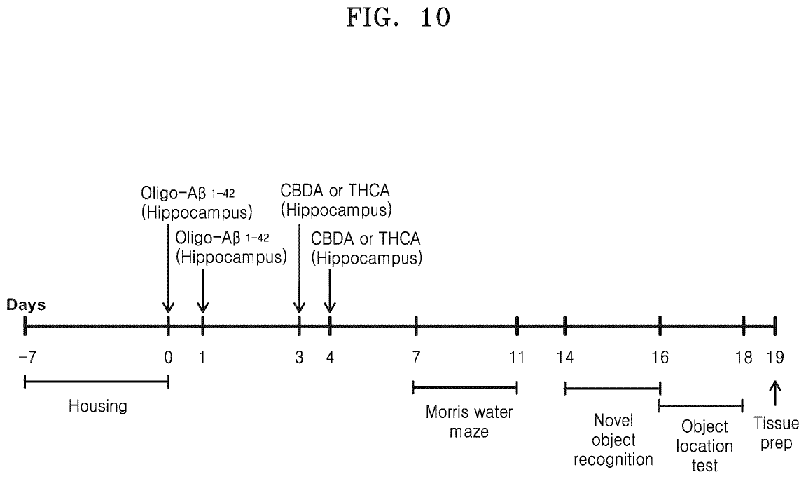

Meanwhile, the compound treatment schedule and experimental progress schedule are shown in FIG. 10.

### 7-2: Morris Water Maze Test (Morris Water Maze, MWM)

To evaluate the cognitive behavioral function of the experimental mice prepared in Example 7-1 above, the Morris water maze test was conducted.

Specifically, a circular tank (diameter 90 cm, height 50 cm, water temperature 22 ±2° C.) was used for the test, and the tank consisted of four quadrants filled with water. An escape platform (diameter 6 cm, height 29 cm) was submerged 1 cm below the water surface in the center of one of the four quadrants. Experimental mice were trained for 4 days to learn and memorize visual cues placed outside the tank indicating the platform location. The swimming paths of the experimental mice were recorded with a camera connected to a video recorder and path tracking software (EthoVision; Noldus Information Technology, Wageningen, The Netherlands). Four tests were conducted daily during the 4-day training period. During each trial, the experimental mice were allowed 60 seconds to find the hidden platform and an additional 30 seconds to remain on the platform. If a mouse did not find the platform within 60 seconds, the mouse was guided to the corresponding platform and allowed to remain on the platform for 30 seconds. The average time it took each experimental mouse to find the platform (average escape latency) was recorded. Probe tests were conducted 4 days later in the same manner without the platform. Each experimental mouse was allowed to move freely for 60 seconds, which was recorded. Videos were analyzed using tracking software (EthoVision; Noldus Information Technology, Wageningen, The Netherlands) to calculate the time spent in the target quadrant area and the number of crossings in the platform area.

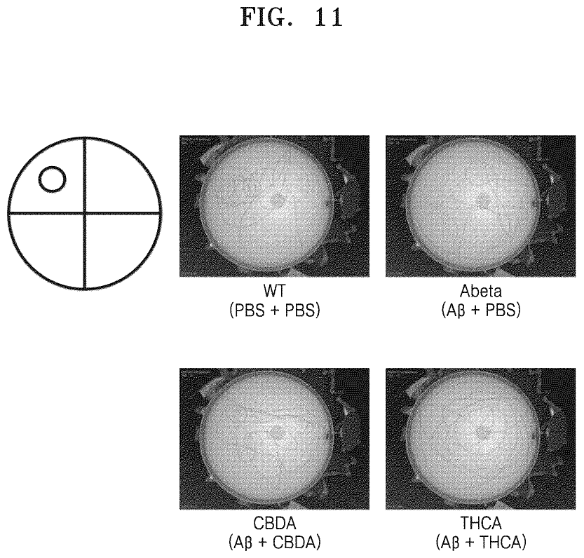

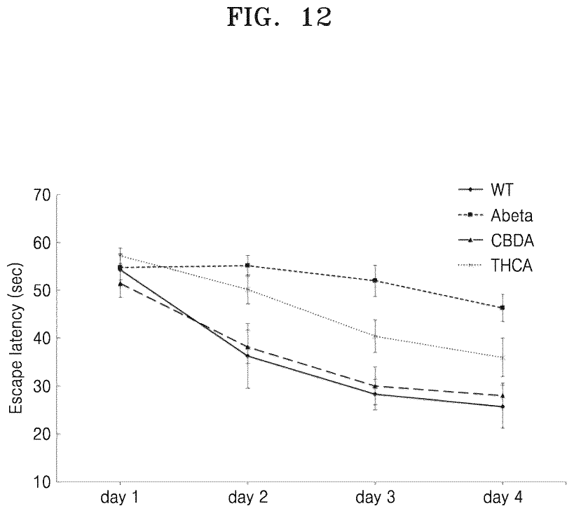

In the Morris water maze test, mice in the Aβ1-42 treated group learned to find the submerged platform during the training session more slowly than mice in the control group. However, in the CBDA or THCA treated group, it was confirmed that learning ability was significantly enhanced compared to mice treated only with Aβ1-42 (FIGS. 11 and 12).

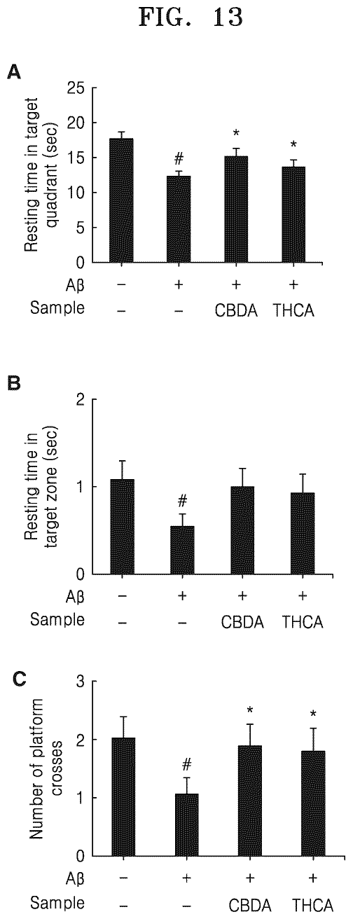

Next, in the probe test conducted on the 5th day, it was confirmed that mice in the Aβ1-42 treated group spent less time in the target quadrant and platform area compared to mice in the control group, but in the CBDA or THCA treated group, they spent more time in the target quadrant and platform area than mice treated only with Aβ1-42 (FIG. 13A and B). In addition, it was confirmed that mice treated with CBDA or THCA also increased the number of times they crossed the platform area compared to mice treated only with Aβ1-42 (FIG. 13C).

Based on the above results, it may be seen that CBDA and THCA may effectively enhance and improve cognitive ability in animal models.

### 7-3: Novel Object Recognition (NOR) and Object Location Test (OLT)

To evaluate the cognitive behavioral functions of the experimental mice prepared in Example 7-1 above, the novel object recognition test (NOR) and the object location test (OLT) were conducted.

First, the Novel Object Recognition Test (NOR) was conducted to examine a natural tendency of the experimental mice to explore novel stimuli. For this purpose, during the habituation session conducted 2 days before the test, the mice were allowed to explore the test environment consisting of an opaque custom-made Plexiglas box (35 cm×45 cm×25 cm) for 10 minutes. The sample object phase was conducted 24 hours later. Two identical white circular cylinders (sample objects) were placed on opposite edges of the test environment, and mice were given access to the objects for 10 minutes. After 4 hours (novel object stage), one of the sample objects in the test environment was replaced with a similarly sized novel object (colored miniature animal), and the mouse was allowed to contact this novel arrangement for 5 minutes. The time when the mouse's nose stayed <1 cm away from the object was considered the time the mouse explored the object, excluding the time when the mouse stood on the object. The identification rate was calculated as the time spent exploring the novel object relative to the time spent exploring the two objects.

Additionally, for the object location test (OLT), it was conducted in the same manner as the novel object recognition test described above, but on the last day of the object location test, one of the two sample objects was moved to a different location and the test was conducted.

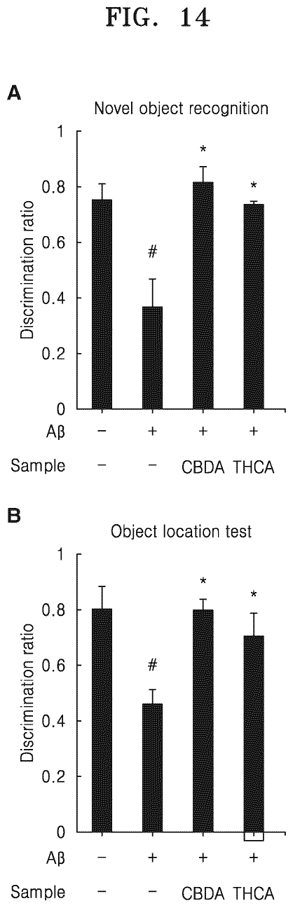

As a result of the novel object recognition test and object location test described above, it was confirmed that the CBDA or THCA-treated group spent more time investigating the novel object or the object moved to a different location than the mice treated with only Aβ1-42, and showed a significantly increased identification rate (FIG. 14).

Based on the above results, it may be seen that CBDA and THCA may effectively enhance cognitive ability in animal models.

### EXAMPLE 8: EVALUATION OF EFFICACY OF CANNABINOIDS IN INHIBITING AMYLOID BETA (Aβ) EXPRESSION LEVELS AT ANIMAL LEVEL

To evaluate the inhibitory efficacy of two compounds (CBDA and THCA) on amyloid beta (Aβ), an Alzheimer's disease pathology marker, for the animal model created in Example 7 above, the following experiments were conducted.

Specifically, hippocampal tissues were acquired on day 19 from the animal models created in Example 7 above (PBS treated group, Aβ1-42 treated group, CBDA treated group, and THCA treated group), and Western blot analysis was conducted on the hippocampal tissues to evaluate the expression levels of APP (Amyloid Precursor Protein), polymeric Aβ, and oligomeric Aβ.

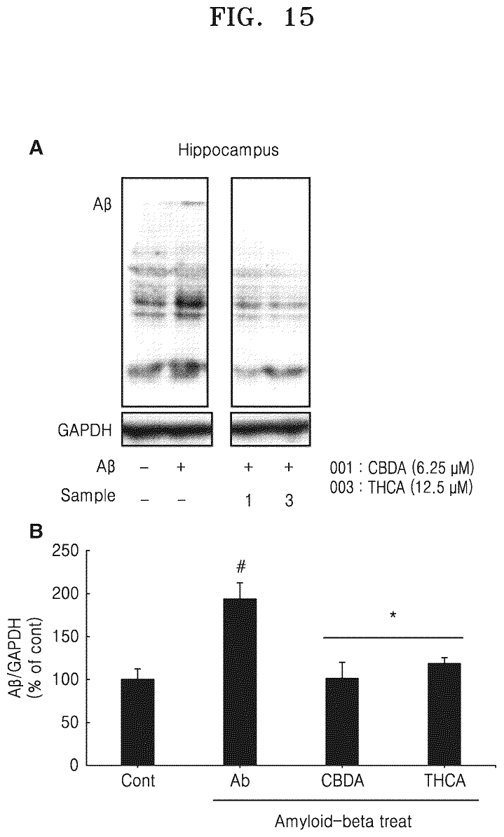

As a result, compared to the Alzheimer's animal model (Aβ1-42 treated group), it was confirmed that the CBDA or THCA treated group significantly reduced the expression level of Aβ (FIG. 15). Therefore, it may be seen that CBDA and THCA may effectively inhibit Aβ, an Alzheimer's disease pathology marker.

### EXAMPLE 9: EVALUATION OF PHOSPHORYLATED TAU (P-TAU) EXPRESSION LEVELS AND PHOSPHORYLATION INHIBITION EFFICACY OF CANNABINOIDS AT ANIMAL LEVEL

To evaluate the efficacy of two compounds (CBDA and THCA) in inhibiting the expression and phosphorylation of phosphorylated tau (p-Tau), an Alzheimer's disease pathology marker, in the animal model created in Example 7 above, the following experiments were conducted.

Specifically, hippocampal tissues were acquired on day 19 from the animal models created in Example 7 above (PBS treated group, Aβ1-42 treated group, CBDA treated group, and THCA treated group), and Western blot analysis was conducted on the hippocampal tissues to evaluate the expression levels of Tau and phosphorylated Tau (p-Tau).

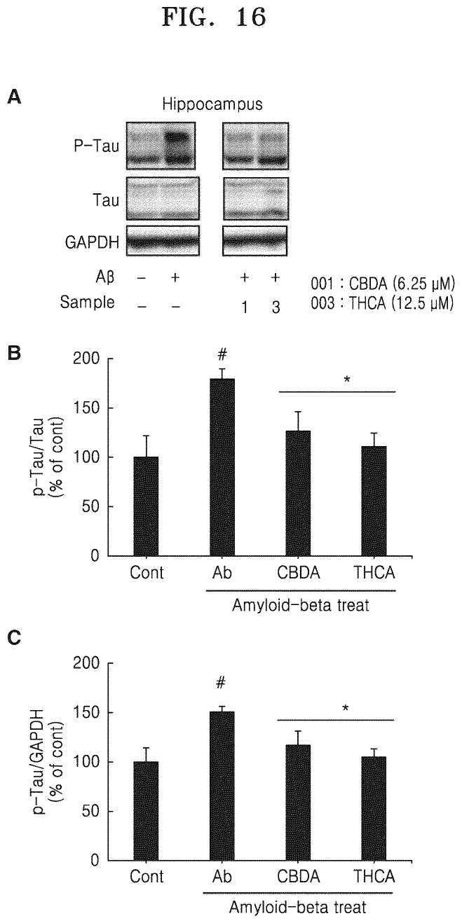

As a result, compared to the Alzheimer's animal model (Aβ1-42 treated group), it was confirmed that the CBDA or THCA treated group significantly reduced the expression level and phosphorylation level of p-Tau (FIG. 16). Therefore, it may be seen that CBDA and THCA may effectively inhibit p-Tau, an Alzheimer's pathology marker.

### EXAMPLE 10: EVALUATION OF EFFICACY OF CANNABINOIDS IN INCREASING BDNF, P-CREB AND P-TRKB EXPRESSION LEVELS AT ANIMAL LEVEL

To evaluate the efficacy of two compounds (CBDA and THCA) in increasing the expression of memory improvement markers BDNF (brain-derived neurotrophic factor), p-CREB (CAMP Response Element-Binding Protein), and p-TrkB (Tyrosine receptor kinase B) for the animal model created in Example 7 above, the following experiments were conducted.

Specifically, hippocampal tissues were acquired on day 19 from the animal models created in Example 7 above (PBS treated group, Aβ1-42 treated group, CBDA treated group, and THCA treated group), and Western blot analysis was conducted on the hippocampal tissues to evaluate the expression levels of BDNF, p-CREB, and p-TrKB.

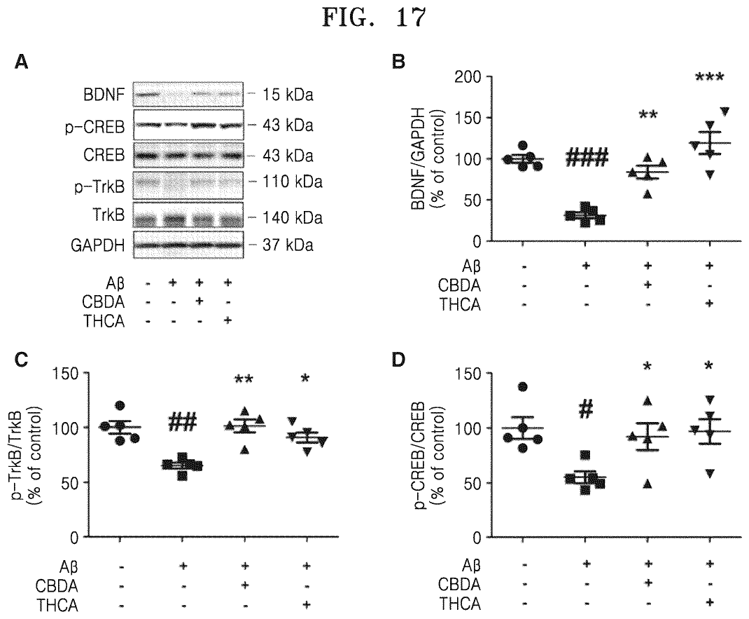

As a result, compared to the Alzheimer's animal model (Aβ1-42 treated group), it was confirmed that the CBDA or THCA treated group significantly increased the expression levels of BDNF, p-CREB, and p-TrKB (FIG. 17). Therefore, it may be seen that CBDA and THCA may effectively restore BDNF, p-CREB, and p-TrKB, which are memory improvement markers.

The foregoing description of the present disclosure is for illustrative purposes only, and one that has ordinary skill in the art to which the present disclosure belongs will understand that the present disclosure may be readily adapted to other specific forms without altering the technical ideas or essential features of the present disclosure. Therefore, the examples described above should be understood in all respects as illustrative and not restrictive.

1. A method of treating or preventing a degenerative brain disease, comprising administering to a subject a pharmaceutical composition comprising cannabinoids as active ingredients.

2. The method of claim 1, wherein the cannabinoids comprise one or more selected from the group consisting of cannabidiolic acid (CBDA), cannabidiol (CBD), tetrahydrocannabinolic acid (THCA), tetrahydrocannabinol (THC), and cannabinol (CBN).

3. The method of claim 1, wherein the cannabinoids comprise cannabidiolic acid (CBDA) and tetrahydrocannabinolic acid (THCA).

4. The method of claim 1, wherein the composition comprises cannabidiolic acid (CBDA) and tetrahydrocannabinolic acid (THCA) in a concentration ratio of 10:0 to 7:3.

5. The method of claim 1, wherein the composition comprises tetrahydrocannabinolic acid (THCA) and cannabidiolic acid (CBDA) in a concentration ratio of 10:0 to 7:3.

6. The method of claim 1, wherein the degenerative brain diseases comprise one or more selected from the group consisting of dementia with Lewy bodies (DLB), multi-infarct dementia (MID), frontotemporal lobar degeneration (FTLD), Pick's disease, corticobasal degeneration (CBD), progressive supranuclear palsy (PSP), Parkinson's disease, Alzheimer's disease, Huntington's disease, amnesia, and memory impairment.

7. The method of claim 1, wherein the composition exhibits one or more characteristics selected from the following:
(a) inhibition of activation or phosphorylation of tau protein;
(b) inhibition of expression, aggregation, or deposition of amyloid beta (Δβ);
(c) inhibition of intracellular Ca²⁺ dyshomeostasis or maintenance of Ca²⁺ homeostasis;
(d) inhibition of nerve or neuronal cell damage;
(e) enhancement/induction of an expression level or activity of brain-derived neurotrophic factor (BDNF);
(f) enhancement/induction of activation or phosphorylation of cAMP response element-binding protein (CREB); and
(g) enhancement/induction of activation or phosphorylation of tyrosine receptor kinase B (TrkB).

8. A pharmaceutical composition for preventing or treating degenerative brain diseases, comprising cannabinoids as active ingredients.
